(19) United States
(12) Patent Application Publication
(10) Pub. No.: US 2024/0131328 A1
(43) Pub. Date: Apr. 25, 2024

(54) ELECTROPORATION DEVICE

(71) Applicant: IGEA S.P.A., Carpi (IT)

(72) Inventors: Roberta FUSCO, Napoli (IT); Francesca DE TERLIZZI, Magliano Veneto (TV) (IT); Ruggero CADOSSI, Carpi (IT); Donata MARAZZI, Carpi (IT)

(73) Assignee: IGEA S.P.A., Carpi (IT)

(21) Appl. No.: 18/470,774

(22) Filed: Sep. 20, 2023

(30) Foreign Application Priority Data
Sep. 23, 2022 (IT) 102022000019635

(51) Int. Cl. A61N 1/32

(52) U.S. Cl. CPC A61N 1/327

(57) ABSTRACT

An electroporation device adapted to perform the following operations: detect the resistance value R_{A,B} between a pair of electrodes placed in a portion of human tissue; calculate, select and store a pair of values of total duration τ(n,t) and voltage V that satisfy the inequality:

$$\frac{σ \cdot {[ ( V / d ) ]}^{2} \cdot τ ( n , t )}{ρ} > A {D}_{R}$$

where:
AD_{R} represents the energy dose that realises a reversible electroporation—the energy dose AD_{R} is a known value stored in the device;
σ is defined as the inverse of resistivity σ=1/ [symbol] ;
d is the spacing of the electrodes;
the resistivity [symbol] =R*S/d where R denotes the measured and stored resistance value and S the section of the electrodes; and
control a power amplifier so that a voltage equal to the voltage V is applied to the electrodes and supplied to the tissue portion for the determined total duration τ.

### CROSS-REFERENCE TO RELATED PATENT APPLICATIONS

This application claims the priority of the Italian Patent Application No. 102022000019635 filed on 23 Sep. 2022, the contents of which are incorporated herein by reference.

### FIELD OF THE ART

The present invention relates to an electroporation device.

### BACKGROUND ART

As is well known, in many surgical and ablative treatments (e.g. aimed at removing malignant or benign tumours), it is important to remove a specific pathological tissue without causing or minimising damage to the surrounding healthy tissue that does not need to be removed.

Electroporation treatments have recently been developed in this field, which are designed to eliminate specific portions of target cell tissue while leaving the surrounding portions unaffected.

As is well known, electroporation treatments involve the application of electric pulses to a tissue by means of electrodes applied to the tissue itself; the electric field generated produces the formation of pores in the cell membrane, causing a change in the cell membrane that eases the flow of organic/inorganic substances (e.g. DNA or drugs) from outside to inside the cell.

These electroporation treatments may be controlled according to the parameters (voltage, waveform, duty cycle, application time, number of applied pulses, etc.) of the electric pulses; by varying these parameters, a reversible or irreversible electroporation can be obtained.

In the first case, the pores of the cell membrane close up following the cessation of pulses and the cells continue to live. In the second case, the high electric field applied to the cells produces pores of such a size that the integrity of the cell membrane cannot be reconstructed and results in cell lysis. Modest thermal effects are present at the surface of the electrodes.

Document WO 2005065284 by DAVALOS, Rafael and RUBINSKY, Boris details the limitations of the electroporation process.

In particular, it is noted how, by controlling the intensity of the applied field and the amplitude of the pulses, it is possible to obtain:
no effect;
a reversible-type electroporation; or
an irreversible electroporation causing cell lysis.

According to the above-mentioned document, irreversible electroporation is obtained by applying pulses with a duration of at least 100 microseconds and a sufficiently high amplitude to induce irreversible damage to the cell membrane causing the death of the cell itself. Reversible pore formation is not expected to be accompanied by lysis or cell death. In case of electric pulses with a high amplitude and sufficient duration, cell lysis is observed. The application of such pulses is accompanied by a high electron flow that causes a state of diffuse inflammation in the tissue.

The object of the present invention is to create a reversible electroporation device adapted to promote the diffusion and passage of drugs or organic molecules (DNA and RNA).

Other earlier documents are: U.S. Pat. No. 7,053,063, WO2020/061,192, US 2021/023,362, US 2019/336,757, U.S. Pat. No. 10,814,129 and US 2020/197,073.

### SUMMARY OF THE INVENTION

The preceding object is achieved by the present invention in that it relates to an electroporation device made according to claim 1.

### BRIEF DESCRIPTION OF THE DRAWINGS

The invention will now be illustrated with reference to the accompanying drawings wherein:

FIG. 1 schematically shows an electroporation device made according to the dictates of the present invention:

FIG. 2 shows a block diagram of the device operations according to the present invention; and

FIG. 3 shows some experimental results obtained with the device in FIG. 1.

### DESCRIPTION OF THE EMBODIMENT

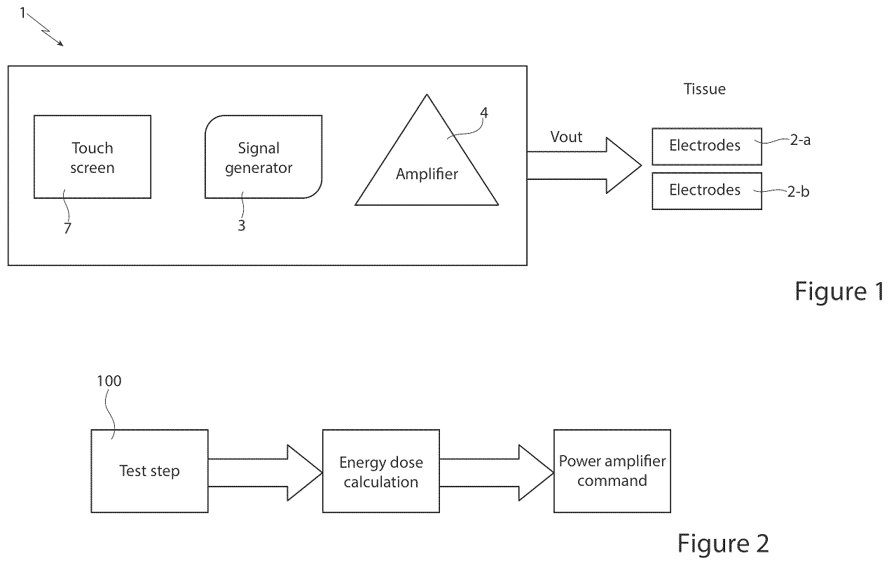

In FIG. 1, an electroporation device which is adapted to provide at least one pair of electrodes 2-a, 2-b with a periodic voltage Vₒᵤₜ having an amplitude and duration which can be controlled as shown hereinafter, is globally referred to as 1. In the non-limiting example shown, a pair of electrodes is present, however, it goes without saying that a different number of electrodes can be used, e.g. four, six or a bipolar electrode.

The electrodes 2-a, 2-b with an elongated rectilinear shape have a length 1, a section S and are arranged facing each other and spaced by a distance d approximately corresponding to the thickness of the area to be treated.

The electroporation device 1 comprises an electronic unit provided with a signal generator 3 that supervises signal generation and a power amplifier 4 that supplies the voltage Vₒᵤₜ to the electrodes 2-a, 2-b.

In use, the pair of electrodes 2-a, 2-b is inserted into a portion of tissue T of a human body to be treated (schematically shown).

The operations performed by the device 1 are the following ones.

Block 100 (Test Step)

The electronic unit commands the following operations.

An initial test pulse (also called “pre-pulse”) Vₜₑₛₜ is generated and supplied to the pair of electrodes 2-a, 2-b and therefore to the tissue T;

Measure the current Iₜₑₛₜ flowing between the pair of electrodes 2-a, 2-b (the current depends on the characteristics of the tissue T);

Detect the resistance value R_{A,B} between the pair of electrodes according to Ohm's law as:

R_{A,B}=(Vₜₑₛₜ)/(Iₜₑₛₜ)

Store the resistance value R_{A,B} detected.

Calculate, according to the resistance value calculated R_{A,B} and according to a maximum current I_{MAX} that can be supplied by the power amplifier 4, the maximum voltage deliverable V_{MAX} as:

V_{MAX}=R_{A,B}×I_{MAX}

The maximum current supplied by the electroporation device I_{MAX} is a function of the technical characteristics of the power amplifier 4 of the electroporation device and it is a known datum, e.g. I_{MAX}=50 amperes.

As a result of the above mentioned operations, the electroporation device operates with the following constraint V<V_{MAX}, i.e. it generates a voltage limited to V_{MAX} such that the current supplied to the electrodes is always less than the maximum deliverable current.

Block 110 Energy Dose Calculation.

Next, the value of the spacing d between the electrodes 2-a, 2-b is supplied to the device 1.

The distance d is supplied by the operator using the electroporation device, e.g. by a command given via touch screen 7 or keyboard.

The block 100 calculates the total duration τ(n,t) (expressed in seconds) of exposure to the local electric field of an electroporation signal comprising several pulses of fixed duration and the local electric field intensity E(k) (expressed in volts per cm) that must be supplied to such a tissue T in order to administer to such tissue T an Absorbed Dose (AD, expressed in J/g) that is greater than the energy dose AD_{R} that achieves a reversible electroporation. The energy dose AD_{R} is a known value stored in the device 1 and is derived experimentally through a series of tests. The threshold value of absorbed energy must be exceeded over the entire target volume to be electroporated. The total duration of exposure to the local electric field T(n,t), in the case of periodic pulses (e.g. a square wave), is given by the number n of pulses multiplied by the time duration t of each pulse. The local electric field intensity E(k), on the other hand, is given by the voltage V applied between two electrodes 2-a, 2-b spaced by the distance d (expressed in cm), i.e. E(k)=V/d.

In particular, the absorbed energy dose AD from the tissue T can be derived according to the following formula:

$$A D = \frac{σ \cdot {[ E ( k ) ]}^{2} \cdot τ ( n , L )}{ρ}$$

Where σ represents the conductivity of the tissue (in Siemens/cm),
And the local electric field intensity (in V/cm),
τ the total duration of the exposure to the local electric field (in sec),
ρ the density of the tissue (as g/cm³).

The absorbed dose AD is expressed in J/g.
σ and ρ are known as properties of the tissue T undergoing electroporation.

In particular:
σ is defined as the inverse of resistivity [symbol] i.e. σ=1/ [symbol] .

The resistivity [symbol] =R*S/d where R denotes the electrical resistance and S the section of the electrodes.

Thus, starting from the resistance calculated with the pre-pulse R_{A,B} and knowing the distance d between the two electrodes and the section of the electrodes 2-a, 2-b we derive σ=d/(R*S).

The electric field E is a function of the voltage V and the distance d between the electrodes 2-a, 2-b i.e. E=V/d.

The formula can therefore be expressed as:

$$A D = \frac{σ \cdot {[ ( V / d ) ]}^{2} \cdot τ ( n , L )}{ρ}$$

The electronic unit calculates and selects a pair of values of total duration τ(n,t) and voltage V that satisfy the inequality:

$$\frac{σ \cdot {[ ( V / d ) ]}^{2} \cdot τ ( n , t )}{ρ} > A {D}_{R}$$

Where as stated above:
AD_{R} represents the energy dose that performs a reversible electroporation—The energy dose AD_{R} is a known value stored in the device 1 and is derived experimentally.
a is defined as the inverse of resistivity σ=1/ [symbol] ;
the resistivity is calculated from the electronic unit as [symbol] =R*S/d where R denotes the electrical resistance (measured in block 100), d the distance between the electrodes (known datum) and S the section (known datum) of the electrodes 2-a, 2-b;

The selection of the voltage torque V and total duration is realised by iterative methods, i.e. by randomly fixing one of the two parameters V and τ(n,t) by finding the other parameter in such a way that the inequality is satisfied. As far as the voltage is concerned, however, the constraint according to which it is below Vmax and above a threshold value is maintained, hence no electroporation effects are obtained.

For example, applying a voltage of V=1000 V and a duration of τ=800 μsec (i.e. 8 pulses with a duration of 100 μsec), an energy dose is obtained which can also be obtained by applying a voltage of 700 V with a duration of 4000 μsec, or by applying a voltage of 500 V with a duration of 8000 μsec.

Block 120 is followed by block 130, which commands the power amplifier for generating the voltage thus determined V and applying this voltage to the electrodes for the determined time τ(n,t).

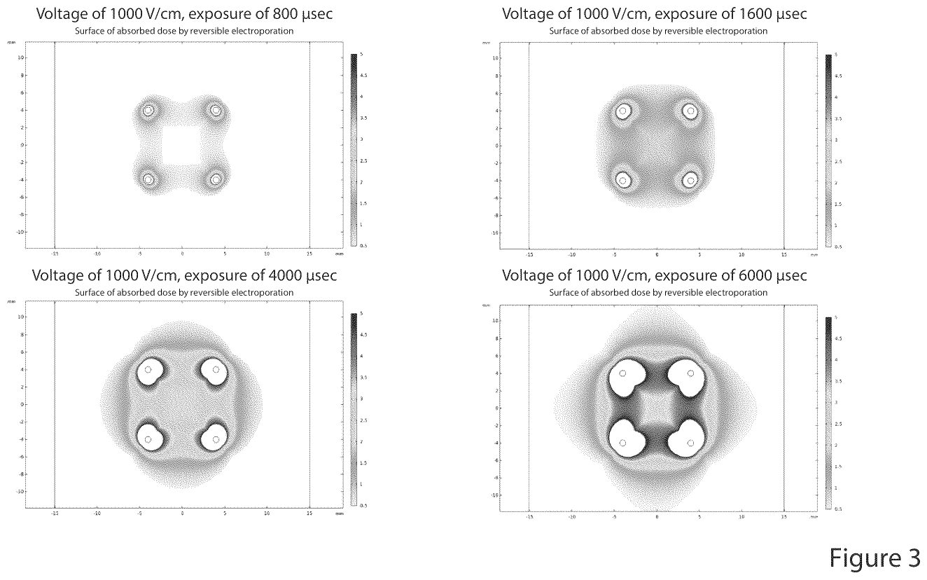

FIG. 3 shows the areas of reversible electroporation obtained from four electrodes with an applied voltage of 1000 V/cm and different values of the total duration τ of exposure to the local electric field.

The size of the electroporated areas in the 2D model are shown in Table 1.

TABLE 1
Reversible electroporation area as the exposure
time to the local electric field changes.

| | | |
|---|---|---|
| Applied potential | Exposure time | Electroporated area |

| | | | | |
|---|---|---|---|---|
| 1000 V | 800 | μsec | 100.5 | mm² |
| 1000 V | 1600 | μsec | 172.1 | mm² |
| 1000 V | 4000 | μsec | 265.8 | mm² |
| 1000 V | 6000 | μsec | 328.6 | mm² |

1. An electroporation device adapted to supply to at least a pair of electrodes (2-a, 2-b) available in a portion of tissue (T) of the human body a periodic voltage Vₒᵤₜ having voltage V and controllable total duration τ;
the electrodes (2-a, 2-b) with elongated rectilinear shape have a section S and are arranged facing and spaced from each other by a distance d;
the electroporation device (1) comprises an electronic unit provided with a signal generator (3) and a power amplifier (4) which supplies the voltage Vₒᵤₜ to the electrodes (2-a, 2-b);
the electroporation device (1) is configured to perform the following operations:
generate at least a first test pulse Vₜₑₛₜ which is supplied to the pair of electrodes (2-a, 2-b) and therefore to the tissue T;
measure the current Iₜₑₛₜ which flows between the pair of electrodes (2-a, 2-b) and which depends on the tissue characteristics (T);
detect the resistance value R_{A,B} between the pair of electrodes according to Ohm's law as:
R_{A,B}=(Vₜₑₛₜ)/(Iₜₑₛₜ)
store the resistance value R_{A,B} detected;
calculate, select and store a pair of values of total duration τ (n,t) and voltage V that satisfy the inequality:
$$\frac{σ \cdot {[ ( V / d ) ]}^{2} \cdot τ ( n , t )}{ρ} > A {D}_{R}$$
where:
AD_{R} represents the energy dose that realises a reversible electroporation—the energy dose AD_{R} is a known value stored in the device (1);
σ is defined as the inverse of resistivity σ=1/ [symbol] ;
d is the spacing of the electrodes;
the resistivity [symbol] =R*S/d where R denotes the measured and stored resistance value and S the section of the electrodes (2-a, 2-b); and control the power amplifier (4) so that a voltage equal to the voltage V is applied to the electrodes and supplied to the tissue portion for the determined total duration τ(n,t).

2. The device according to claim 1 wherein selection of the voltage torque V and total duration is obtained by means of iterative methods, namely by fixing at random one of the two parameters V and τ (n,t), finding the other parameter so that the inequality is satisfied.

3. The device according to claim 2 wherein the electroporation device (1) is furthermore configured to carry out the following operations:
calculate, according to the resistance value calculated R_{A,B} and according to a maximum current I_{MAX} that can be supplied by the power amplifier (4), the maximum voltage deliverable V_{MAX} as:
V_{MAX}=R_{A,B}×I_{MAX}
the maximum current supplied by the electroporation device I_{MAX} is a function of the technical characteristics of the power amplifier (4) of the electroporation device and is a known datum; limit the voltage delivered to a value below V_{MAX} so that the current supplied to the electrodes is below the maximum deliverable current.

4. The device according to claim 3 wherein selection of the voltage torque V and total duration is achieved by selecting a voltage below V_{MAX} and above a threshold value whereby electroporation effects are not obtained.
